(19) United States
(12) Patent Application Publication
(10) Pub. No.: US 2008/0161404 A1
(43) Pub. Date: Jul. 3, 2008

(54) Bicalutamide for Delivering Increasing Steady State Plasma Levels

(75) Inventors: Kevin Joseph Carroll, Cheshire (GB); Thomas Meredydd Morris, Cheshire (GB); Ian Derek Cockshott, Cheshire (GB); Bo-Eric Persson, Copenhagen (DK); Paul Alfred Dickinson, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Södertälje (SE)

(21) Appl. No.: 11/816,428

(22) PCT Filed: Feb. 21, 2006
(86) PCT No.: PCT/GB06/00594

§ 371 Date: Nov. 2, 2007

Related U.S. Application Data
(60) Provisional application No. 60/655,517, filed on Feb. 23, 2005.

(51) Int. Cl. A61K 31/165; A61P 35/00

(52) U.S. Cl. 514/618

(57) ABSTRACT

The present invention relates to a method of treating a metastatic prostate cancer patient by administering to said patient an effective amount of a bicalutamide (4′-cyano-α′,α′,α′-trifluoro-3-(4-fluorophenylsulphonyl)-2-hydroxy-2-methylpropiono-m-toluidide) containing formulation capable of delivering at least a mean steady state plasma level of (R)-bicalutamide enantiomer of 40 μg/ml; and to bicalutamide containing products and formulations capable of delivering the at least mean steady state plasma level of (R)-bicalutamide enantiomer of 40 μg/ml to a patient in need thereof.

Bicalutamide, a non-steroidal anti-androgen, is the racemate of 4′-cyano-α′,α′,α′-trifluoro-3-(4-fluorophenylsulphonyl)-2-hydroxy-2-methylpropiono-m-toluidide. Bicalutamide is known by the AstraZeneca trade name CASODEX™. EP-100172 discloses 4′-cyano-α′,α′,α′-trifluoro-3-(4-fluorophenylsulphonyl)-2-hydroxy-2-methylpropiono-m-toluidide (named in EP-100172 as 4-cyano-3-trifluoromethyl-N-(3-p-fluorophenylsulphonyl-2-hydroxy-2-methylpropionyl)aniline) as the 8^{th} compound listed in the table in Example 6. The corresponding structure is shown in formula I:

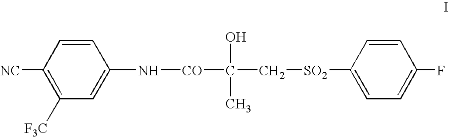

Bicalutamide can be used to combat prostate cancer, and the properties and usefulness of bicalutamide as an anti-androgen have been reviewed in B J A Furr et al., Urology, 1996, 47 (Suppl. 1A), 13-25, and G J C Kolvenbag et al., Urology, 1996, 47 (Suppl. 1A), 70-79.

Bicalutamide is used in conventional oral tablet form at a dosage of 150 mg once daily as monotherapy for the treatment of early (localised or locally advanced) non-metastatic prostate cancer in men. It is used at a dosage of 50 mg once daily in combination with leutinising hormone-releasing hormone analogue or surgical castration for the treatment of advanced prostate cancer.

The bioavailability of the bicalutamide to the patient is determined to a certain extent by the dissolution rate and solubility of the drug in the gastrointestinal (GI) tract, which affects absorption across mucosal membranes in the GI tract. The relative bioavailability of bicalutamide for a series of formulations can be assessed by determining the area under the curve (AUC) of a graph of plasma bicalutamide concentration v. time elapsed since administration of the bicalutamide. As a consequence of sub-optimal rates of dissolution and degree of solubility of the drug, the maximum systemic exposure achievable after dosing the conventional tablet is limited, such that at conventional tablet doses in excess of 150 mg, there is a significant reduction in bicalutamide bioavailability. The inventors have confirmed the earlier finding by Kolvenbag et al. (The Prostate 34:61-72, 1998) that, with conventional Casodex™ tablets at dosages at or above 300 mg/day, no further significant increase in systemic exposure is achievable. However, no efficacy data for these higher dosages of Casodex™ is disclosed in Kolvenbag et al. (supra).

The current standard treatment for M1 patients is castration (surgical or medical orchiectomy). However, this is associated with serious side effects such as impairment of cognitive function, impairment of sexual function, impairment of physical capacity and a reduction in bone mineral density that may lead on to osteoporotic complications (Iversen et al., J Urol 164(5):1579-1582, 2000; and, Sieber et al., J Urol 171(6 Pt 1):2273-2276, 2000) leading to diminished quality of life. Although it is generally accepted that quality of life measures are better for patients on Casodex™ rather than castration, therapy with 150 mg Casodex™ has not proved as successful as castration in terms of overall survival in men with metastatic disease (Tyrrell et al., Eur Urol. 33: 447-456, 1998). There is therefore a need in the art for improved ways of treating M1 patients that have an overall survival chance as good as, and preferably better than, patients receiving castration, without the diminished quality of life issues identified above.

Currently Casodex™ 50 mg is licensed for the management of advanced disease in combination with castration therapy, while Casodex™ 150 mg is licensed as monotherapy for the management of patients with non-metastatic disease. In Japan, the dose is 80 mg in all indications. The mean plasma concentration of R-bicalutamide in patients administered these doses are approximately 9 μg/ml, 19 μg/ml and 22 μg/ml respectively (Tyrrel et al., Eur Urol. 1998;33(1):39-53, 1998; Cockshott et al., Eur Urol. 18 (Suppl 3):10-7, 1990). As disclosed in Example 1 herein, at doses above 150 mg the increments in plasma exposure does not rise linearly with dose, and at unit doses above 300 mg, no further increase in mean plasma steady state exposure are seen. Thus, using the conventional Casodex™ formulation, regardless of dose above 300 mg/day, the maximum mean steady state exposure appears to be in the range of 31-36 μg/ml.

The present invention arises from the observation of an increased survival benefit in metastatic prostate cancer patients treated with higher than normal dosages of Casodex™ capable of delivering mean steady state plasma levels of the (R)-bicalutamide greater than that obtained with conventional 150 mg Casodex™. This facet of bicalutamide therapy provides, for the first time, the ability to treat a sub-group of prostate cancer patients, those with metastatic prostate cancer (classed as M1), with sufficient benefit to compete with or outdo the benefits of castration (medical or surgical), using a bicalutamide formulation capable of delivering a mean steady state plasma level of (R)-bicalutamide enantiomer equal to or greater than 40 micrograms per ml.

Accordingly, the present invention relates to a method of treating metastatic prostate cancer patients by administering to a patient in need thereof a bicalutamide (4′-cyano-α′,α′,α′-trifluoro-3-(4-fluorophenylsulphonyl)-2-hydroxy-2-methylpropiono-m-toluidide) containing formulation capable of delivering at least a mean steady state plasma level of (R)-bicalutamide enantiomer of 40 μg/ml.

Further aspects of the invention include the use of bicalutamide, preferably the (R)-enantiomeric form thereof, in the manufacture of a pharmaceutical product for treating metastatic prostate patients, preferably as a monotherapy.

According to one aspect of the invention there is provided a method of treating metastatic prostate cancer patients by administering to a patient in need thereof an effective amount of a bicalutamide (4′-cyano-α′,α′,α′-trifluoro-3-(4-fluorophenylsulphonyl)-2-hydroxy-2-methylpropiono-m-toluidide) containing formulation capable of delivering at least a mean steady state plasma level of (R)-bicalutamide enantiomer of 40 μg/ml.

In alternate embodiments the formulation is capable of delivering at least 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 100 μg/ml, and 110 μg/ml mean steady state plasma level of (R)-bicalutamide enantiomer. It will be appreciated that these are mean steady state values and that there will be inter-patient variability and a lag period from initial administration until the blood levels have reached a steady state level.

In view of the heterogeneity of the patient population, it will be apparent to the person skilled in the art, that different persons on the same dosage form of a drug may exhibit different steady state plasma levels. Accordingly, the values referred to herein (e.g. 40 μg/ml mean steady state plasma levels) are average values taken from at least 20 patients, preferably at least 100 patients, and more preferably from at least 1000 patients. The mean steady state plasma levels are calculated for each dosage of a particular formulation.

The patient is preferably a human, e.g. an adult male, but the treatment of other mammals is also contemplated.

It is well established that the bicalutamide active moiety is the (R)-enantiomeric form of bicalutamide. The (S)-enantiomer is rapidly cleared relative to (R)-enantiomer, the latter having a plasma elimination half-life of about 1 week. Steady state plasma concentrations of the (R)-enantiomer, of approximately 22 μg/ml are observed during daily administration of Casodex™ 150 mg. At steady state, the predominantly active (R)-enantiomer accounts for approximately 99% of the total circulating enantiomers.

The term “metastatic prostate cancer” patient or “M1” patient refers to a patient who has spread of the prostate cancer into distant structures or organs or the bony skeleton.

According to another aspect of the invention there is provided a method of treating a patient suffering from metastatic prostate cancer comprising administration to said patient bicalutamide in an amount and in a formulation capable of delivering mean steady state plasma concentrations of the (R)-bicalutamide of at least 40 μg/ml.

According to a further aspect of the invention there is provided a method of treating metastatic prostate cancer comprising maintaining steady state plasma concentrations of (R)-bicalutamide of at least 40 μg/ml for an effective length of time, in a patient in need thereof.

By the term “effective amount” is meant the dosage which is sufficient to bring about the desired effect, such amounts can be determined via clinical trials by the person skilled in the art without undue experimentation or inventive effort. The “effective length of time” can also be determined by the person skilled in the art, for example by the physician looking after the individual patient being treated.

The term “treating prostate cancer” as used herein means treating, alleviating or palliating such condition, suppressing the growth of cancerous tissue and thus providing an increase in survival time or quality of life.

As noted above, because of the plateau effect, the conventional formulation of Casodex™ is incapable of delivering the required plasma levels of (R)-bicalutamide regardless of dosage administered. However, the inventors have discovered that solubility-enhancing formulations, such as the solid dispersion formulations disclosed in WO 02/067893, WO 02/080902 and WO 03/032950, incorporated herein by reference, are capable of delivering the plasma levels of (R)-bicalutamide required by the present invention.

As defined herein, a particular formulation is “solubility enhancing” if it exhibits dissolution above 30% at any of the following time points: 5, 10, 15, 30, 45 and 60 minutes, when a 75 mg bicalutamide (R- or R/S) containing formulation is placed in 1.7 litres of 0.1 M potassium dihydrogen orthophosphate at pH 6.8, with stirring at 37° C. The drug:solution (weight:volume) ratio can be adapted accordingly.

A formulation that does not meet this functional criterion is defined herein as a “conventional formulation” or “non-solubility enhancing”.

The invention could therefore also be performed using other solubility-enhancing formulations of bicalutamide. There are many alternate solubility-enhancing methods known in the art. For example, in addition to solid dispersion systems, techniques such as: lipidic formulation, cyclodextrin complexation, particle size reduction, super disintegrants, micellation, emulsions etc. have been proposed and shown to enhance the solubility of a drug. The textbook entitled “Water Insoluble Drug Formation”—editor Rong Liu. (2000), describes some of the different solubility enhancing techniques known. For example:

| | |
|---|---|
| complexation | (chapter 6) |
| solubilization using co-solvent approach | (chapter 7) |
| emulsions and microemulsions | (chapter 8) |
| micelles | (chapter 9) |
| liposomes | (chapter 10) |
| alternate salt forms | (chapter 11) |
| prodrugs | (chapter 12) |
| particle size reduction (Incl. micronization and | (chapter 13) |
| nanosuspensions) | |
| solid dispersion technology | (chapter 14) |
| alteration of the solid state, polymorphs, solvates etc. | (chapter 15) |

The paper by Pinnamaneni et al. “Formulation approaches for orally administered poorly soluble drugs” Pharmazie. 57(5):291-299 (2002), also reviews the various alternate solubility-enhancing methods available to the person skilled in the art.

Examples of recent patents/applications directed to some of these alternate solubility-enhancing formulations of drugs include: U.S. Pat. No. 6,569,463 (Lipocine), U.S. Pat. No. 6,294,192 (Lipocine), WO 01/028505 (Lipocine), WO 04/050057 (Technologies Biolactis Inc.), WO 03/043602 (DDS Tech. Co. Ltd), WO 02/030397 (Lipotec SA), each incorporated herein by reference. Each and any of these solubility enhancing formulation methods could be utilised in the therapeutic methods of the invention.

The inventors have however, made another surprising finding. The (R)-enantiomeric form of bicalutamide is approximately 3-fold more soluble than racemic bicalutamide present in the Casodex™ formulation (potentially equating to a 6-fold enhancement in exposure). This has meant that whilst racemic bicalutamide in the conventional formulation is incapable of delivering the required steady state plasma levels required for the methods of the present invention, the formulation of (R)-enantiomeric bicalutamide in a conventional (non-solubility enhancing) formulation, such as the one for conventional Casodex™, may well achieve the plasma blood levels of (R)-enantiomer needed to carry out the invention.

A conventional/non-solubility enhancing formulation is defined above. Typically, a non-solubility enhancing formulation would contain excipients to aid manufacture of the dosage form and release of the drug substance in the body, such as fillers, binders, disintegrants, lubricants, flow aids. It may also contain surfactants to aid the wetting and dissolution rate of the drug substance.

Bicalutamide, 4′-cyano-α′,α′,α′-trifluoro-3-(4-fluorophenylsulphonyl)-2-hydroxy-2-methylpropiono-m-toluidide, can exist in distinct (R)- and S-enantiomeric forms. The (R)-enantiomer is the (−) isomer and is the pharmacologically active compound in vivo. For further details of the enantiomers, reference is made to Tucker and Chesterton, J. Med. Chem. 31, pp 885-887 (1988).

The terms (R)-enantiomer, (R)-bicalutamide and (R)-bicalutamide enantiomer are used interchangeably herein.

The chemical synthesis of racemic 4′-cyano-α′,α′,α′-trifluoro-3-(4-fluorophenylsulphonyl)-2-hydroxy-2-methylpropiono-m-toluidide is described in U.S. Pat. No. 4,636,505, and this disclosure is incorporated herein by reference. The (R)-enantiomer may be obtained by chromatographic separation of diastereomeric esters of chiral acids. For example, the (R)-enantiomer may be prepared by chromatographic separation using chiral chromatography. Another method involves resolution of the carboxylic acid precursor, 3-(4-fluorophenyl)-2-hydroxy-2-methylpropanoic acid, by fractional crystallisation of diastereomeric salts with chiral amines. The Tucker and Chesterton reference cited above discloses the chromatographic separation of the (R)-and (S)-enantiomers from racemic 4′-cyano-α′,α′,α′-trifluoro-3-(4-fluorophenylsulphonyl)-2-hydroxy-2-methylpropiono-n-toluidide. The method involves the chromatographic separation of (R)-camphanoyl esters of the racemate and their hydrolysis and oxidation to the (R)- and (S)-enantiomers. This disclosure is incorporated herein by reference specifically to provide an illustration of a method of obtaining the bicalutamide enantiomers for use in the present invention. Other methods (including asymmetric synthesis, e.g. U.S. Pat. No. 6,583,306—Nobex) will, however, be evident to the skilled addressee using routine techniques for the preparation of enantiomers.

The present invention extends to the use of racemate and/or the (R)-enantiomeric form.

As noted above, bicalutamide is currently administered as the racemate. However, it is well established that the (R)-enantiomer contains substantially all of the anti-androgenic activity. Accordingly, in one embodiment of the compound, formulation or dose, ≧50%, ≧60%, ≧65%, ≧70%, ≧80%, ≧85%, ≧90%, ≧95%, ≧98% or ≧99% or thereabout of the 4′-cyano-α′,α′,α′-trifluoro-3-(4-fluorophenylsulphonyl)-2-hydroxy-2-methylpropiono-m-toluidide (bicalutamide) is provided in the form of the (R)-enantiomer. In a preferred embodiment, 100%, or substantially 100%, of the 4′-cyano-α′,α′,α′-trifluoro-3-(4-fluorophenylsulphonyl)-2-hydroxy-2-methylpropiono-m-toluidide is provided in the form of the (R)-enantiomer. By “substantially 100%” we mean that the 4′-cyano-α′,α′,α′-trifluoro-3-(4-fluorophenylsulphonyl)-2-hydroxy-2-methylpropiono-m-toluidide is provided as the pure (R)-enantiomer, or there is a trace (<1%) of the S-enantiomer present.

The person skilled in the art of pharmaceutical formulation is capable of designing a suitable formulation for administering the drug. The compounds or compositions for use according to the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Formulation in general is described in Chapter 25.2 of Comprehensive Medicinal Chemistry, Volume 5, Editor Hansch et al, Pergamon Press 1990.

A composition for use in the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or acetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

In embodiments that use racemic bicalutamide, it is preferred that an enhanced bioavailability formulation be adopted. For example, the bioavailability of racemic bicalutamide (as well as (R)-enantiomer) can be enhanced by formulating the drug as a solid dispersion formulation. Thus, in one embodiment the non-steroidal anti-androgen drug is prepared as a formulation comprising the drug in solid dispersion with an enteric polymer having a pKa from 3 to 6, or PVP. Such formulations are disclosed in WO 02/067893, WO 02/080902 and PCT/GB02/04621, incorporated herein by reference.

In one embodiment, the enteric polymer is selected from hydroxypropyl methylcellulose acetate succinate (HPMCAS), hydroxpropyl methylcellulose acetate phthalate, hydroxypropyl methylcellulose acetate, hydroxypropyl methylcellulose succinate, a methacrylic acid copolymer, polyvinyl acetate phthalate (PVAP), cellulose acetate phthalate (CAP), methylcellulose acetate phthalate, ethyl cellulose acetate phthalate, hydroxypropyl cellulose acetate phthalate, hydroxypropyl methylcellulose phthalate (HPMCP), cellulose propionate phthalate, hydroxypropyl cellulose butyrate phthalate, hydroxypropyl cellulose acetate phthalate succinate, hydroxypropyl rnethylcellulose trimellitate, cellulose acetate trimellitate (CAT), methylcellulose acetate trimellitate, ethyl cellulose acetate trimellitate, hydroxypropyl cellulose acetate trimellitate, iydroxypropyl methylcellulose acetate trimellitate, hydroxypropyl cellulose acetate trimellitate succinate, cellulose propionate trimellitate, cellulose butyrate trimellitate, cellulose acetate terephthalate and cellulose actetate isophthalate.

The use of the term “hydroxypropyl methylcellulose phthalate polymer”, or HPMCP, is known to the skilled reader for classifying a group of polymers which share the same basic structural features and include such polymers as: hypromellose phthalate; methylhydroxypropylcellulosi phthalas; cellulose, hydrogen 1,2-benzenedicarboxylate, 2-hydroxypropyl methyl; as well as commercially available polymers HP-55™, HP-55S™ and HP-50™ (available from Shin-Etsu Chemical Industry Co., Ltd., Japan or appointed distributors).

Preferably the hydroxypropylmethylcellulose phthalate polymer has a molecular weight (Mw) from 20 kDa to 200 kDa, e.g. from 80 kDa to 130 kDa. In one embodiment, the Mw is less than 150 kDa, or less than 100 kDa. HP-50, HP-55 and HP-55S are polymers known in the literature and widely used as an enteric coating for oral formulations. HP-55 has a Mw 84 kDa. HP-55S has a Mw of 132 kDa. HP-50 has a Mw 78 kDa. HP-50 is soluble at pH≧5, whereas HP-55 and HP-55S are soluble at pH≧5.5. In one embodiment, the non-steroidal anti-androgen is in a solid dispersion with at least one polymer selected from HP-50, HP-55 and HP-55S. Thus, it is contemplated that a mixture of two or more of these HPMCP polymers can be used.

In one embodiment, the non-steroidal anti-androgen is (R)-enantiomeric bicalutamide in solid dispersion with HP-55S polymer.

In an alternate embodiment the (R)-enantiomeric form of bicalutamide is formulated in a non-solubility enhancing/conventional tablet form, such as one containing lactose monohydrate, sodium starch glycolate, povidone and magnesium stearate. An alternative example would be a tablet containing calcium phosphate, microcrystalline cellulose, hydroxypropyl methylcellulose, croscarmellose sodium and stearic acid. Further examples of the components of a non-solubility enhancing formulation are given in example 3.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, and esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 30 mg to 600 mg of active agent compounded with an appropriate and convenient amount of excipients which may vary from about 5 to about 98 percent by weight of the total composition. Dosage unit forms will generally contain about 50 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The bicalutamide compound according to the various aspects of the invention it will be administered in a daily dosage form capable of providing at least 40 μg/ml steady state plasma levels of (R)-bicalutamide. Typically, unit dosage forms will contain about 60 mg to 400 mg of bicalutamide.

According to a further aspect of the invention there is provided a pharmaceutical formulation or product comprising bicalutamide formulated in an amount and form capable of delivering at least a mean steady state plasma level of (R)-bicalutamide enantiomer of 40 μg/ml when administered to man.

According to another aspect of the invention there is provided the use of bicalutamide in the manufacture of a pharmaceutical product for treating metastatic prostate cancer patients, characterised in that the product is in an amount and in a form capable of delivering at least a mean steady state plasma level of (R)-bicalutamide enantiomer of 40 μg/ml when administered to man. The ‘amount’ refers to the amount of drug (in either racemic or R-enantiomeric form) present in the formulation. The ‘form’ refers to the actual formulation, such as a solubility enhancing formulation (e.g. solid dispersion with an polymer) or conventional formulation. Thus, the amount of R-enantiomeric bicalutamide in any formulation will be less than that required if the drug was in racemic form, also, the amount of R-enantiomeric form of drug will be less in a solubility enhancing formulation compared to a conventional formulation.

The inventors have discovered that doses of Casodex (racemic bicalutamide in a conventional tablet form) in the range of 300 mg to 600 mg per day for an appropriate length of time provides equivalent treatment for metastatic prostate cancer patients, in terms of overall survival, as castration. Thus, according to another aspect of the invention there is provided a formulation comprising 300 mg bicalutamide. This could either be a single 300 mg unit dose (e.g. a single tablet) or multiple tablets of a lower dose (e.g. 2×150 mg). According to a further aspect, there is a method of treating a metastatic prostate cancer patient by administering to said patient at least a 300 mg/day amount of racemic bicalutamide, or the equivalent amount of R-enantiomeric bicalutamide, for a suitable length of time.

The invention will be further described by way of the following non-limiting examples and accompanying figures in which:

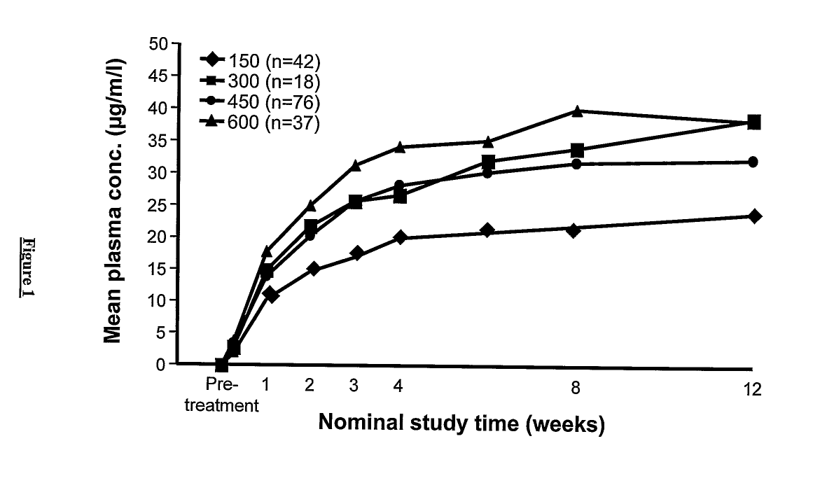

FIG. 1—shows geometric mean (R)-bicalutamide steady-state plasma concentration (Cₛₛ) for daily racemic bicalutamide doses of 150-600 mg over a 12-week period.

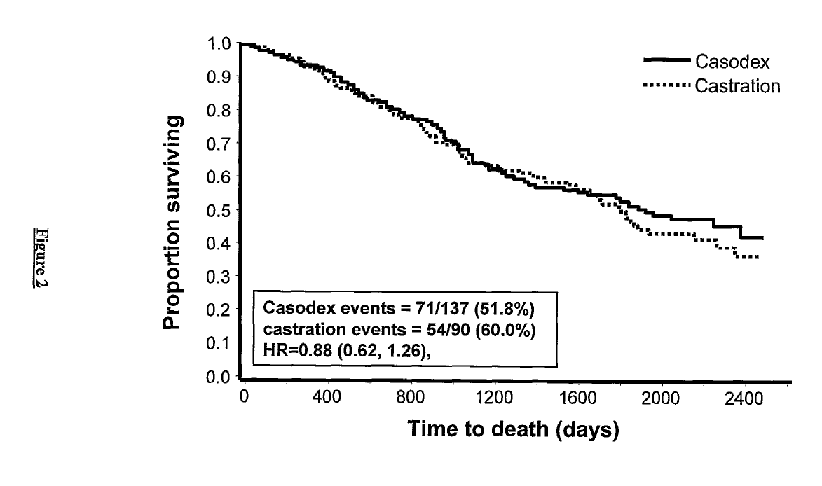

FIG. 2—shows survival rates for patients receiving high doses of bicalutamide (450-600 mg) and those in the castration treatment group (bicalutamide events 71/137 [51.8%] versus castration events 54/90 [60.0%]; hazard ratio [HR]=0.88 [95% CI 0.62,1.26]).

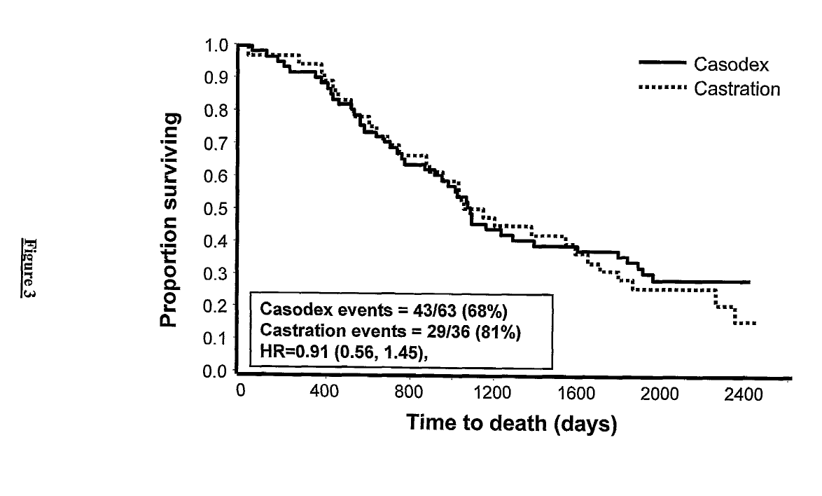

FIG. 3—shows survival rates for M1 patients receiving high doses of bicalutamide (450-600 mg) and those in the castration treatment group (bicalutamide events 43/63 [68.3%] versus castration events 29/36 [80.6%]; HR=0.91 [95% CI 0.56,1.45]).

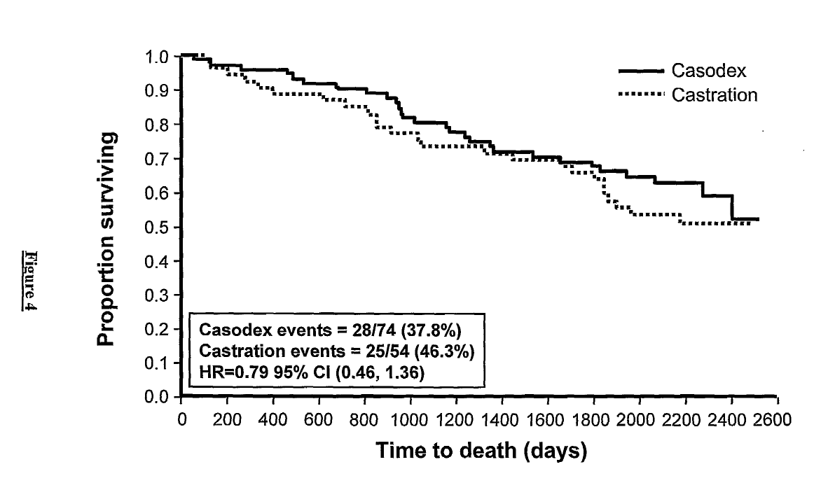

FIG. 4—shows survival rates for M0 patients receiving high doses of bicalutamide (450-600 mg) and those in the castration treatment group (bicalutamide events 28/74 [37.8%] versus castration events 25/54 [46.3%]; HR=0.79 [95% CI 0.46,1.36]).

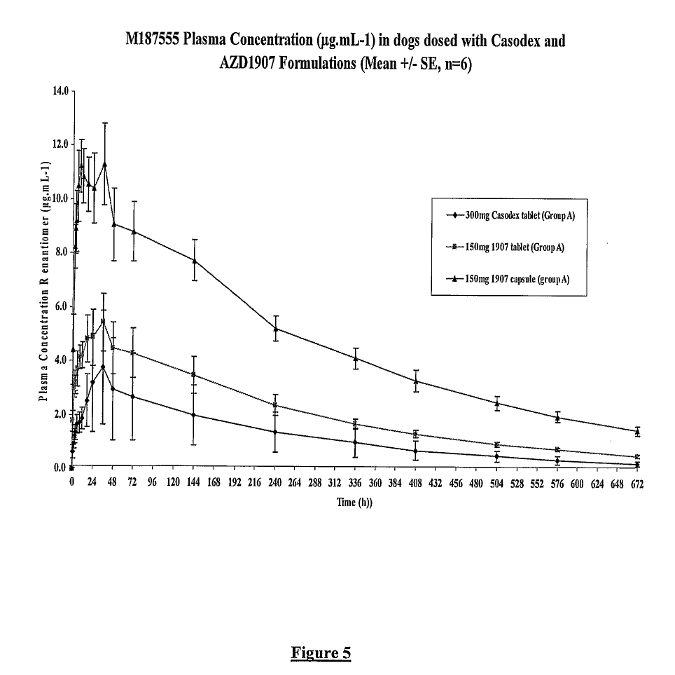

FIG. 5—shows mean plasma concentration profiles for R-enantiomer seen after oral dosing of Casodex™ tablets (300 mg; bottom line, diamond), R-enantiomer tablets (150 mg; middle line square) or R-enantiomer solid dispersion capsules (150 mg; top line, triangle) in Group A dogs.

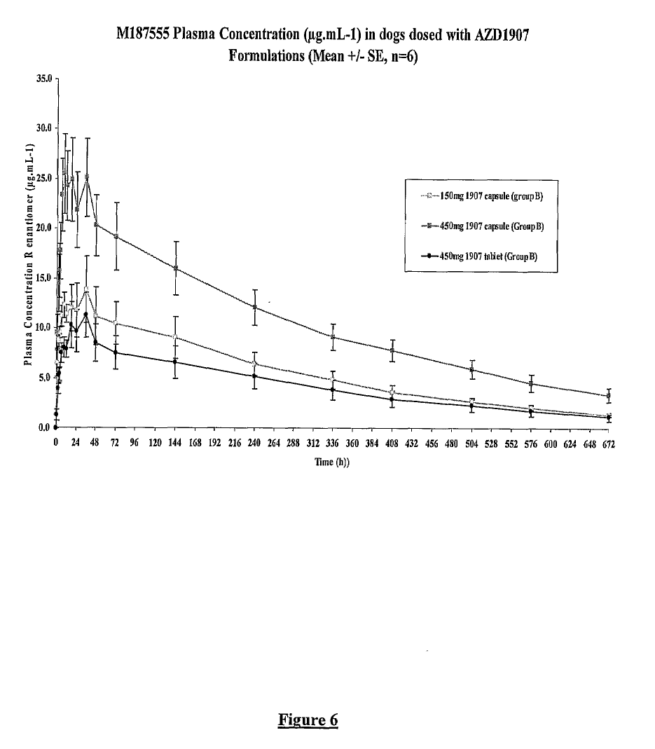

FIG. 6—shows mean plasma concentration profiles for R-enantiomer seen after oral dosing of R-enantiomer tablets (450 mg; bottom line) or R-enantiomer solid dispersion capsules (150 mg or 450 mg, middle and top lines respectively) in Group B dogs.

### EXAMPLES

### Example 1

This first clinical study/Example was designed to assess the efficacy, pharmacokinetics and safety of higher doses (above 150 mg) of bicalutamide given once daily as monotherapy to patients with M0 or M1 prostate cancer.

### Patients

Patients had histologically or cytologically confirmed M0 (T3 or T4) or M1 prostate cancer and a PSA level ≧20 ng/mL.

### Study Design and Treatment

Twenty patients were initially recruited into a non-randomised phase to assess the tolerability of bicalutamide 300 mg (2×150 mg Casodex™ tablets) orally, once daily. Tolerance was assessed in these patients after 6 weeks of treatment and, if acceptable, subsequent patients were randomised (on a 1:1 randomisation basis) to either bicalutamide or castration (bilateral orchiectomy or goserelin acetate 3.6 mg depot (Zoladex™) every 28 days) therapy. Tolerability was similarly assessed at 450 mg/day (3 tablets) and 600 mg/day (4 tablets).

Although the study was designed to allow dose escalations of bicalutamide up to 900 mg/day, after assessment of the first 3 doses it became apparent that absorption of bicalutamide had become saturated, evidenced by similar Cₛₛ and t_{1/2} over the 300-600 mg dose range. The safety profile was similar across this dose range. Consequently, dose escalation ceased at 600 mg.

Patients continued to receive bicalutamide or goserelin acetate until: serious adverse event, death, objective disease progression, unwillingness, inability or refusal to continue randomised treatment, addition of systemic treatment for prostate cancer without objective progression, or loss to follow-up. After objective disease progression, patients were followed up every 3 months for survival and/or adverse event data.

A total of 248 patients were randomised to treatment. Of these patients, 139 had M0 prostate cancer and 109 had M1 prostate cancer.

In total, 21 patients received bicalutamide 300 mg, 95 patients were randomised to receive 450 mg (although 3 patients received no treatment), and 42 patients were randomised to receive bicalutamide 600 mg. Of the 90 patients randomised to castration, 82 patients received medical castration and 8 patients had a bilateral orchiectomy. All four treatment groups were well balanced with respect to their demographics and disease status on entry to the study.

All patients were evaluable for safety and efficacy with the exception of the three patients who were randomised to bicalutamide 450 mg but received no treatment.

The mean duration of treatment was 27.6 (range 1.9-75.6), 30.0 (range 2.0-78.7) and 29.2 (range 0.5-73.5) months for patients receiving bicalutamide 300, 450 and 600 mg, respectively, and 33.5 (range 0-78.2) months for patients in the castration treatment group.

### Pharmacokinetic Assessments

The pharmacokinetic endpoints were the steady-state concentrations (Cₛₛ) and the plasma elimination half-life (t_{1/2}) of (R)-bicalutamide. (S)-bicalutamide Cₛₛ levels were also determined.

Blood samples for pharmacokinetic analyses were collected before starting treatment and on Days 1, 7, 14, 21, 28, 42, 56, and 84 after the first dose of bicalutamide. Plasma samples were stored at −20° C. until analysis. Bicalutamide compound was isolated from plasma by solvent extraction (expand) and fractionation after chromatography. The fraction containing the bicalutamide was subjected to a second chromatographic system where the R- and S-enantiomers of bicalutamide are separated. Quantification of the enantiomers was achieved using ultra-violet absorbance.

Method: Assays were performed using 0.5 ml plasma aliquots. A calibration series was prepared by spiking aliquots of control human plasma with standard solutions of racemic bicalutamide. The concentrations of the standards are therefore x with respect to bicalutamide and x/2 with respect to each enantiomer. A further control compound (internal standard) was added to each sample, along with 1.5 ml of pH 7 0.05M phosphate buffer (Electronic Instruments Ltd.) and 6 mls of ethyl acetate. The samples were mixed and then centrifuged and a 5 ml aliquot of the ethyl acetate layer (upper layer) was removed and evaporated to dryness in a polypropylene tube. The residue was re-dissolved in 400 μl of water:acetonitrile (70:30 v/v). The bicalutamide and internal standard compounds were detected by their U.V. absorbance at 270 nm following elution of 200 μl of the reconstituted residue from 100 mm×4.6 mm i.d. 5 μm Hypersil ODS chromatography column; HPLC solvent—acetonitrile:water (50:50 v/v). Quantification of bicalutamide in the test samples was achieved by comparison of the peak height ratios of the compound to internal standard in the test samples with those in the calibration series.

The bicalutamide peak isolated from the above chromatographic system was collected into a glass tube using a fraction collector. The fraction was evaporated to dryness and reconstituted in 400 μl of an HPLC eluent; 0.02 M pH 7 phosphate buffer; acetonitrile (85:15 v/v). 200 μl of the reconstituted fraction was then injected onto the chiral chromatographic system.

R- and S-bicalutamide enantiomers were detected by their U.V. absorbance at 270 nm following their elution from a 150 mm×4.6 mm i.d. stainless steel column packed with ‘Ultron’ ES-OVM (ovomucin phase; icrom); this was fitted with a guard column packed with 5 μm chiral-AGP (J.T Baker Inc.). Quantification of R- and S-bicalutamide enantiomers was achieved by comparison of the peak height and/or area of the compound peak in the test samples, with those of the calibration series.

Haematological, biochemical and endocrine assessments were performed before starting treatment, at Weeks 4, 8 and 12 and every 12 weeks thereafter (only to Week 24 for endocrine assessments).

The primary efficacy endpoint was the change in serum PSA levels after 12 weeks' treatment with bicalutamide or castration.

Kaplan-Meier plots of time to death were used to compare the effect of high doses (450-600 mg) of bicalutamide or castration on survival rates of patients with advanced prostate cancer. Results are presented as for both the overall patient population and also by disease stage (M0 or M1). See FIGS. 2, 3 and 4.

### Results

Pharmacokinetic data were derived for 18, 82 and 34 patients who received bicalutamide 300, 450 and 600 mg, respectively.

TABLE 1

| | 300 mg | 450 mg | 600 mg |
|---|---|---|---|
| Steady state plasma | 35 | 31 | 36 |
| conc. geometric | | | |
| mean (μg/ml) | | | |
| Patients with data | 18 | 82 | 34 |

For 150 mg dose, steady state concentration is 21 μg/ml

Plasma concentrations of the active (R)-bicalutamide enantiomer from Day 2 to Week 12 of dosing with bicalutamide 150, 300, 450 or 600 mg/day are shown in FIG. 1.

The geometric means Cₛₛ were similar for all three doses of bicalutamide, suggesting that systemic exposure had reached a plateau. Similar results were observed for the inactive (S)-bicalutamide enantiomer.

Geometric means t_{1/2} of (R)-bicalutamide were also similar across the three bicalutamide dose groups. Although there was a slight trend for decreasing t_{1/2} with increasing bicalutamide dose, the range of values was similar across the three groups. During the study, 139/245 patients (56.7%) died. Thirty-nine patients died while they were on study therapy, 15 of these 245 patients died from prostate cancer.

### Efficacy

From baseline to Week 4, patients receiving bicalutamide showed greater percentage reductions in geometric mean PSA values compared with those in the castration group. At Week 12, however, the percentage decrease in PSA was similar across all treatment groups (93, 95, 96 and 95% reduction for patients receiving bicalutamide 300, 450 and 600 mg, and castration, respectively).

With a median of 5 years' follow up, survival rates were similar among patients receiving high doses of bicalutamide (450-600 mg) and those in the castration treatment group (bicalutamide events 71/137 [51.8%] vs castration events 54/90 [60.0%]; hazard ratio [HR]=0.88 [95% CI 0.62,1.26]). See FIG. 2.

In patients with M1 prostate cancer, there was no difference in survival between those receiving high doses of bicalutamide (450-600 mg) and those in the castration treatment group (bicalutamide events 43/63 [68.3%] vs castration events 29/36 [80.6%]; HR=0.91 [95% CI 0.56,1.45]). See FIG. 3.

Similarly, in M0 patients, no differences in survival were observed between those receiving high doses of bicalutamide (450-600 mg) and those in the castration treatment group (bicalutamide events 28/74 [37.8%] vs castration events 25/54 [46.3%]; HR 0.79 [95% CI 0.46,1.36]). See FIG. 4.

### Discussion

Pharmacokinetic assessments showed that 300, 450 and 600 mg doses of bicalutamide all gave similar plasma steady state concentrations indicating that systemic exposure had reached a plateau. Previous dose ranging studies have shown a linear increase in systemic exposure to bicalutamide for doses up to about 200 mg (Tyrrell et al 1998a). In the present study, plasma concentrations of (R)-bicalutamide at doses of ≧300 mg (31-36 μg/mL) were approximately 50% higher than those observed previously with bicalutamide 150 mg (21.6 μg/mL) and approximately 15% higher than those observed with bicalutamide 200 mg (28.9 μg/mL) (Tyrrell et al., Eur Urol. 33:39-53, 1998) indicating non-linearity in Cₛₛ at doses above 200 mg.

The t_{1/2} values in the present study, however, were similar to those observed previously with lower bicalutamide doses (6.25-8.88 days at bicalutamide doses of 10-200 mg) (Tyrrell et al 1998a) indicating no trend in (R)-bicalutamide elimination pharmacokinetics with doses up to 600 mg and suggesting that the elimination processes were not saturated.

This suggests that the observed non-linearity in Cₛₛ with dose was a consequence of a decreasing proportion of the drug being absorbed as the bicalutamide dose increased. Pharmacokinetic studies in animals have shown that absolute bioavailabilty of bicalutamide declines with increasing doses, probably because of its low aqueous solubility (Cockshott, Clin. Pharmacokinet. 43(13):855-878, 2004).

After a median of 5 years follow-up, survival rates in patients receiving high doses of bicalutamide (450-600 mg) were equivalent to those seen with castration therapy. Analysis of survival in patients with different stages of prostate cancer (M0 or M1) indicated that bicalutamide 450-600 mg was equivalent to castration in both patient groups. These findings potentially opens up the use of a Casodex™ tablets at doses of 300 mg or above in the treatment of metastatic prostate cancer via monotherapy.

In a previous study, bicalutamide 150 mg was shown to provide similar survival benefits to castration in patients with M0 prostate cancer (Iversen et al 2000).

In contrast to patients with M0 prostate cancer, results from previous studies have suggested that bicalutamide 150 mg is not as effective as castration in M1 patients (Tyrrell et al 1998b).

The present study suggests that higher doses of bicalutamide may provide a similar survival outcome to castration in patients with M1 prostate cancer. The 300 mg, 450 mg and 600 mg doses of bicalutamide deliver higher steady state plasma levels of R-bicalutamide (31-36 μg/mL) than those observed previously with current 150 mg (21.6 μg/mL). There is thus a correlation between increased steady state plasma levels of R-bicalutamide and survival benefit in M1 patients. This suggests that if greater than 36 μg/mL steady state plasma levels of R-bicalutamide could be delivered to M1 patients, even greater efficacy and survival benefit, possibly exceeding that obtained by castration, may be possible.

Bicalutamide has been shown to provide quality of life benefits over castration, particularly in terms of sexual interest and physical capacity, which can be important considerations for some men (Iversen et al 2000; Tyrrell et al 1998b). Furthermore, bicalutamide 150 mg offers an additional advantage in terms of maintaining bone mineral density while castration often results in progressive loss in bone mineral density (Sieber et al 2004; Smith et al 2004).

This therefore also suggests that higher dose bicalutamide could be an option for M1 patients who do not wish to undergo castration.

### Example 2

### Solubility Studies

### A. Solubility in Phosphate Buffer Solutions

The aqueous buffer solutions were prepared as given in the table (Table 2) and the pH confirmed by measurement before use.

TABLE 2

| | pH 1.3 | pH 3.2 | pH 6.2 | pH 8.3 |
|---|---|---|---|---|
| Sodium Dihydrogen | 0.3 | 2.89 | 2.51 | 0.09 |
| Orthophosphate (g) | | | | |
| Disodium Hydrogen | — | — | 0.54 | 2.74 |
| Orthophosphate (g) | | | | |
| Phosphoric acid (ml) | 1.1 | 0.08 | — | — |
| Total Volume (ml) | 200 | 200 | 200 | 200 |

Excess racemic bicalutamide or R-bicalutamide was added to 20 ml of each buffer solution. The mixtures were mechanically stirred for 24 hours at a temperature of about 23° C. It was confirmed that undissolved solid still remained in each buffer solution after this period to ensure as far as possible that saturated solutions had been prepared. The final pH of the solutions was measured.

The concentration of racemic bicalutamide or R-bicalutamide in each of the buffers was measured using HPLC (150×4.6 mm RP18 3.5 μm Column; 350:650 Acetonitrile: water eluent; 1.0 ml/min flow rate; 270 nm detection wavelength; 10 μl injection volume).

The solubility of racemic bicalutamide and R-bicalutamide at pH 1.3, 3.2, 6.2 and 8.3 at about 23° C. are shown in Table 3.

TABLE 3
Solubility data for racemic bicalutamide and R-bicalutamide
in 0.1M aqueous Phosphate Buffer Solutions

Solubility mg/ml at 23° C.

| pH | R-Bicalutamide | Bicalutamide | Ratio |
|---|---|---|---|
| 1.3 | 0.0069 | 0.0025 | 2.8 |
| 3.2 | 0.0067 | 0.0024 | 2.8 |
| 6.2 | 0.0065 | 0.0022 | 3.0 |
| 8.3 | 0.0061 | 0.0021 | 2.8 |

### Discussion

The data show that at pH 1.3, 3.2, 6.2 and 8.3, the solubility of R-bicalutamide is approximately three times that of racemic bicalutamide.

Although the solubility of the single R-enantiomer is 3 times that of the racemate it is possible in vivo that this will translate to a 6-fold improvement in exposure of the R-enantiomer. This is because the dose of the racemate is double that of the single enantiomer for the same dose of single enantiomer. The point at which non-linearity in exposure is observed is a consequence of the dose to solubility ratio and thus the potential benefit is 6 fold rather than 3 fold.

### B. Dissolution of Racemic Bicalutamide and R-Bicalutamide Formulations

Dissolution studies have been performed on formulations containing racemic bicalutamide or R-bicalutamide. These comprised a conventional tablet formulation containing 80 mg racemic bicalutamide (Casodex 80 mg tablets); a conventional tablet formulation containing 150 mg R-bicalutamide (R-bicalutamide tablet); a solubility-enhanced formulation containing 75 mg of bicalutamide (bicalutamide 75 mg capsules) and a solubility-enhancing formulation containing 75 mg R-bicalutamide. The solubility enhancing formulations were solid dispersions of drug and HP-55s enteric polymer, prepared according to WO 02/067893 and WO 03/032950. The conventional tablet containing R-bicalutamide was prepared according to Example 3 herein.

Standard dissolution apparatus was used (USP II, paddles at 100 rpm, 37° C., 0.1M potassium dihydrogen orthophosphate at pH 6.8). The volume of dissolution medium was adjusted to take account of the strength of the tablets so that 100% dissolution of the drug would yield approximately the same concentration of drug in the dissolution medium. For each formulation, one dosage unit was placed in each dissolution pot and the stirrer started. A 10 ml sample was taken from each pot after 15, 30 and 45 minutes and the amount of dissolved drug in each aliquot determined by HPLC (12.5×4.6 mm 3 u RP18 column; 650:200:150, water:tetrahydrofuran:acetonitrile eluent; 270 nm detection wavelength; 1.5 ml/min flow rate; 10 μl injection). The results are shown in Table 4.

TABLE 4
Dissolution results for conventional and solubility enhanced formulations of
racemic bicalutamide (termed bicalutamide) and R-bicalutamide (termed R-bic)

| | | | | Concentration | |
|---|---|---|---|---|---|
| | | | | of R- or R/S | |
| | Dissolution | Volume | Time | bicalutamide | % |
| Formulation | Medium | Used | minutes | (mg/ml) | dissolution |
| Bicalutamide | 0.1M | 1800 ml | 15 | 0.019 | 45.6 |
| 75 mg capsule | potassium | | 30 | 0.036 | 86.4 |
| solubility | dihydrogen | | 45 | 0.039 | 93.9 |
| enhanced | orthophospahate | | | | |
| formulation | at pH 6.8 | | | | |
| R-bic 75 mg | 0.1M | 1700 ml | 15 | 0.043 | 98 |
| capsule | potassium | | 30 | 0.047 | 107 |
| solubility | dihydrogen | | 45 | 0.047 | 107 |
| enhanced | orthophospahate | | | | |
| formulation | at pH 6.8 | | | | |
| Bicalutamide | 0.1M | 1800 ml | 15 | 0.0029 | 6.6 |
| 80 mg tablet | potassium | | 30 | 0.0034 | 7.6 |
| conventional | dihydrogen | | 45 | 0.0036 | 8.2 |
| formulation | orthophospahate | | | | |
| | at pH 6.8 | | | | |
| R-bic 150 mg | 0.1M | 3400 ml | 15 | 0.007 | 16.0 |
| tablet | potassium | | 30 | 0.0088 | 20.0 |
| conventional | dihydrogen | | | | |
| formulation | orthophospahate | | | | |
| | at pH 6.8 | | | | |

As shown in Table 3 above, at 23° C. and at pH 6.2, the solubility of R-enantiomer bicalutamide is approximately 0.0065 mg/ml and that of racemic bicalutamide about 0.0022 mg/ml. We expect a conventional formulation to exhibit similar increase in solubilization. Indeed, bearing mind the temperature differences, the results of the dissolution studies shown in Table 4 support this conclusion.

### Discussion

For the conventional formulations dissolution at pH 6.8 was incomplete being only 21.6% for the R-bicalutamide tablets and 8.2% for the racemic bicalutamide tablets. The dissolution of the tablets containing R-bicalutamide was approximately three times that of those containing racemic bicalutamide at 15, 30 and 45 minutes. This finding agrees well with the increased solubility of R-bicalutamide over racemic bicalutamide that is reported above.

In contrast the enhanced solubility formulations demonstrated essentially complete dissolution at pH 6.8 after 45 minutes. Interestingly, the enhanced formulation containing R-bicalutamide also showed complete dissolution at 15 and 30 minutes, in contrast to the formulation containing racemic bicalutamide which gave a lower dissolution of 46% and 86% after 15 and 30 minutes respectively. This indicates that whereas the formulation has successfully enhanced the solubility of both products, racemic bicalutamide, the compound with lower solubility, dissolutes more slowly in the enhanced formulation.

### Example 3

### Preparation of Pharmaceutical Forms of Bicalutamide.

A conventional tablet formulation may typically contain some of the following excipients, along with the drug substance:
Fillers (also known as diluents and compression aids) including lactose, mannitol, microcrystalline cellulose, calcium phosphate, calcium carbonate.
Binders including carboxymethylcellulose sodium, povidone, hydroxypropyl methylcellulose, hydroxypropyl cellulose, pregelatinized starch, gelatin.
Disintegrants including sodium starch glycolate, croscarmellose sodium, starch, crospovidone.
Lubricants including magnesium stearate, stearic acid, polyethylene glycol, talc.
Flow aids such as colloidal silica dioxide.

A conventional tablet formulation may be manufactured by direct compression, or by using granulation methods. A direct compression process would involve mixing or blending of the components in suitable equipment followed by compression into solid dosage forms using an appropriate tablet machine.

Granulation processes are used to improve the properties of the powdered components, for example, flow properties or compression properties. Wet granulation involves the addition of a granulation fluid e.g. water or a binder solution, to the powdered components during mixing. This results in agglomeration of the powders into granules. The granules are then typically dried, passed through a mill and blended with a lubricant. The blended granules are then compressed on an appropriate tablet machine.

Dry granulation is similar to the above process with the exception that the granules are formed by compression process rather than by the addition of a granulating fluid. This may be by roller compaction or slugging processes. The resultant granules are milled blended and compressed to form a tablet.

The 150 mg R-bicalutamide tablets of Example 2 and 4 comprise the following ingredients:

| | | |
|---|---|---|
| R-bicalutamide pure bulk drug micronised | 150 | mg/tab |
| Lactose Monohydrate | 183 | mg/tab |
| Sodium starch glycolate | 22.5 | mg/tab |
| Povidone | 15 | mg/tab |
| Magnesium Stearate | 4.5 | mg/tab |

Batch product was manufactured by a wet granulation process, i.e. drug was mixed with lactose & sod starch glycollate; aqueous solution of povidone was sprayed on with mixing to granulate; the granules were then dried in a fluid bed drier; the magnesium stearate was then added and the dried granules were then put though a mill, blended in a tumble blender. Finally, the tablets were prepared by compression.

### Example 4

### Enhanced Bioavailability With R-Bicalutamide in a Conventional Tablet.

A study in dogs was performed to assess the change in bioavailability when changing from the racemic bicalutamide to the R-enantiomer in a conventional tablet formulation and in an enhanced solubility solid dispersion of the drug with the enteric polymer HP-55s. Three formulations were dosed at 2 dose levels of the R-enantiomer. The formulations were:
Formulation: 1. 150 mg Casodex™ tablet (racemate)
   2. 150 mg R-bicalutamide tablet
   3. 75 mg R-bicalutamide solid dispersion capsule

Single doses of 150, 300 or 450 mg were administered orally to fasted 2 to 6 year old Beagle dogs weighing 10-19 kg, on each of three dosing days. In total, 12 dogs were dosed, split into two dosing groups of 6 dogs. A 3 way crossover design was utilised with dogs in Group A receiving either 2×150 mg Casodex™ tablets, 1×150 mg R-enantiomer tablet and 2×75 mg R-enantiomer solid dispersion capsules at each dosing, while dogs in Group B received either 2×75 mg R-enantiomer solid dispersion capsules, 6×75 mg R-enantiomer solid dispersion capsules and 3×150 mg R-bicalutamide tablets at each dosing. Each dosing day was 4 weeks apart

Dogs were fed about 400 g of Special Diet Services Laboratory Diet A each day, and allowed water ad libitum.

2 ml whole blood in lithium heparin tubes were taken from the jugular vein immediately prior to dosing and at 1, 2, 3, 4, 6, 9, 12, 18, 24, 36, 48, 72 and 144 hours; and at 10, 14, 17, 21, 24 and 28 days (the 28 day sample from the first and second dosing was also the pre-dose sample for dosing day 2 and 3 respectively). The blood was centrifuged at 3000 rpm for 15 minutes and plasma removed into plain blood tubes and the plasma stored at −20° C. until analysis.

Plasma concentrations of the R enantiomer were determined using a high-pressure liquid chromatography coupled to tandem mass spectrometry after plasma extraction. The assay was specific for the R enantiomer and exhibited acceptable accuracy and precision. Plasma concentration time data was manipulated using standard techniques. Relative exposure was determined based on the area under the plasma concentration time curve from zero to infinity (AUC) and maximum plasma concentration (Cₘₐₓ) (M. Rowland and T. N. Tozer (1995) Clinical Pharmacokinetics: Concepts and Applications. 3rd Ed. Lippincott, Williams and Wilkins. Media, Pa., USA). Plasma concentrations were corrected for pre-dose concentration prior to calculation of AUC and Cₘₐₓ. Statistical significance was assessed at the 95% level on untransformed data using paired t-test for within group comparisons and unpaired t-test for across group comparisons.

Mean plasma concentration profiles for R-enantiomer seen after oral dosing of Casodex™ tablets, R-enantiomer tablets or R-enantiomer solid dispersion capsules in Group A and B dogs are shown in FIGS. 5 and 6 (in which R-bicalutamide is referred to as 1907). It can be seen that dosing the same amount of R-enantiomer as either Casodex™ tablets, R-enantiomer tablets (AZD 1907 tablets) or R-enantiomer solid dispersion capsules (AZD 1907 capsules) does not produce the same exposure. R-enantiomer solid dispersion capsules producing greater exposure than R-enantiomer tablet, which in turn produced greater exposure than Casodex™ tablets. The relative exposure and whether they are significantly different are summarised in Table 5. In all cases AUC was significantly different and as AUC is directly related to steady-state concentration it can be concluded that these differences in AUC will lead to difference in Cₛₛ in the clinic. At the 450 mg dose of R-enantiomer tablets (Group B dogs, FIG. 6) it can be seen that although R-enantiomer plasma exposure from the R-enantiomer tablet increased relative to that seen for 150 mg dose in Group A (FIG. 5), the exposure was similar and not statistical different to that seen for R-enantiomer solid dispersion capsules at 150 mg (Table 5). At 450 mg dose of R-enantiomer solid dispersion capsules the exposure increased to about two fold of that seen from the 150 mg dose.

These data show that R-enantiomer tablets and solid dispersion capsules are able to produce R-enantiomer Css in the clinic that are greater than that which can be produced by Casodex™ tablets, up to at least three to four fold higher for R-enantiomer tablet and eight to nine fold higher for the R-enantiomer solid dispersion capsules.

Thus, it is expected that a suitable unit dosage form of an R-enantiomer conventional tablet or as an enhanced solubility formulation can be prepared that is capable of delivering >40 ug/ml mean steady state plasma levels of R-bicalutamide suitable for use in the present invention.

TABLE 5
Relative exposure of R-enantiomer in plasma after dosing of Casodex ™ tablets,
R-enantiomer tablets and R-enantiomer solid dispersion capsules.

| | | Relative | Relative | Statistical | Statistical |
|---|---|---|---|---|---|
| | | Increase | Increase | Significant | Significant |
| Dog Set | Comparison | in AUC | in Cₘₐₓ | on AUC | on Cₘₐₓ |
| Group A | 300 mg Casodex ™ tablet | ×1.8 | ×1.4 | Yes | No |
| | <150 mg R-enantiomer | | | | |
| | tablet | | | | |
| | 300 mg Casodex ™ tablet | ×4.3 | ×3.2 | Yes | Yes |
| | <150 mg R-enantiomer | | | | |
| | solid dispersion capsules | | | | |
| | 150 mg R-enantiomer | ×2.4 | ×2.2 | Yes | Yes |
| | tablet <150 mg R- | | | | |
| | enantiomer solid | | | | |
| | dispersion capsules | | | | |
| Group B | 450 mg R-enantiomer | ×1.3 | ×1.3 | No | No |
| | tablet <150 mg R- | | | | |
| | enantiomer solid | | | | |
| | dispersion capsules | | | | |
| | 450 mg R-enantiomer | ×2.5 | ×2.4 | Yes | Yes |
| | tablet <450 mg R- | | | | |
| | enantiomer solid | | | | |
| | dispersion capsules | | | | |
| | 150 mg R-enantiomer | ×2.0 | ×1.9 | Yes | Yes |
| | solid dispersion capsules | | | | |
| | <450 mg R-enantiomer | | | | |
| | solid dispersion capsules | | | | |
| Across | 150 mg R-enantiomer | ×2.1 | ×2.1 | Yes | Yes |
| group | tablet <450 mg R- | | | | |
| comparisons | enantiomer tablet | | | | |
| | 150 mg R-enantiomer | ×1.1 | ×1.2 | No | No |
| | solid dispersion capsules | | | | |
| | <150 mg R-enantiomer | | | | |
| | solid dispersion capsules | | | | |

### Example 5

### Study of High Dose R-Bicalutamide in Metastatic Disease

The following is a proposed clinical trial study design to test R-bicalutamide:HP55s solid dispersion formulation as a monotherapy in treating metastatic prostate cancer patients.

### Patient Numbers and Power Calculation

Based on preliminary estimates of survival and variability, and allowing for a drop out rate of 10%, then 2750 patients would need to be randomised and included in the analysis.

### Patient Population

Patients with:
Histologically proven prostate cancer
Documented metastatic disease
Not yet been treated with any hormonal therapy
Life expectancy greater than 6 months

### Study Drug

The dose of R-bicalutamide:HP55s solid dispersion formulation required to provide a mean steady state plasma exposure of at least 40 μg/ml, would be identified from dose ranging studies.

### Study Design

Open, randomised, multi-centre, multinational phase III study

Mean survival assumed to be 4 ys

Study duration >6 ys

Visits every 3 months for first year and then every 6 months for life thereafter

Safety labs, PSA, adverse events collected at all visits

Pharmacokinetic analysis at 1^{st} year visits

Initial survival analysis at 1.5 ys post randomisation of last patient

1. A pharmaceutical product comprising bicalutamide formulated in an amount and form capable of delivering at least a mean steady state plasma level of (R)-bicalutamide enantiomer of 40 μg/ml when administered to man.

2. The product according to claim 1, comprising bicalutamide in racemic form.

3. The product according to claim 1, comprising bicalutamide in R-enantiomeric form.

4. The product according to claim 1, wherein the bicalutamide is formulated in a non-solubility enhancing formulation.

5. The method according to claim 1, wherein the bicalutamide is formulated in a solubility enhancing formulation.

6. The method according to claim 5, wherein the solubility enhancing formulation is a solid-dispersion formulation.

7. A pharmaceutical formulation comprising bicalutamide formulated in an amount and form capable of delivering at least a mean steady state plasma level of (R)-bicalutamide enantiomer of 40 μg/ml when administered to a patient in need thereof.

8. (canceled)

9. A method of treating a metastatic prostate cancer patient by administering to a patient in need thereof an effective amount of a bicalutamide (4′-cyano-α′,α′,α′-trifluoro-3-(4-fluorophenylsulphonyl)-2-hydroxy-2-methylpropiono-m-toluidide) containing formulation capable of delivering at least a mean steady state plasma level of (R)-bicalutamide enantiomer of 40 μg/ml.

10. The method according to claim 9, wherein the formulation is capable of delivering mean steady state plasma level of (R)-bicalutamide enantiomer selected from the group consisting of at least: 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 100 μg/ml and 110 μg/ml.

11. A method of treating metastatic prostate cancer comprising maintaining steady state plasma concentrations of (R)-bicalutamide of at least 40 μg/ml for an effective length of time, in a patient in need thereof.

12. The method according to claim 9, wherein the formulation comprises bicalutamide in racemic form.

13. The method according to claim 9, wherein the formulation comprises bicalutamide in R-enantiomeric form.

14. The method according to claim 9, wherein the formulation is a non-solubility enhancing formulation.

15. The method according to claim 9, wherein the formulation is a solubility enhancing formulation.

16. The method according to claim 15, wherein the solubility enhancing formulation is a solid-dispersion formulation.
